SEQUENCE LISTING (6 sequences)

SEQ ID NO: 1 - DNA, 62 bp, Artificial Sequence
  Synthetic oligonucleotide
ctgacatgcccaggcatgtcttgacatgcccaggcatgtcttgacatgcccaggcatgtcta

SEQ ID NO: 2 - DNA, 70 bp, Artificial Sequence
  Synthetic oligonucleotide
catggactgtacgggtccgtacagaactgtacgggtccgtacagaactgtacgggtccgtacagatctag

SEQ ID NO: 3 - DNA, 3522 bp, Artificial sequence
  Synthetic oligonucleotide
ggcctaactggccggtacctgagctcgctagcctcgaggatatcaagatctggcctcggcggccaagcttggcaatccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 4 - DNA, 3556 bp, Artificial sequence
  Synthetic oligonucleotide
ggcctaactggccggtacctgacatgcccaggcatgtcttgacatgcccaggcatgtcttgacatgcccaggcatgtctagatctggcctcggcggccaagcttggcaatccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 5 - DNA, 3598 bp, Artificial sequence
  Synthetic oligonucleotide
ggcctaactggccggtacctgacatgcccaggcatgtcttgacatgcccaggcatgtcttgacatgcccaggcatgtctagatctggcctcggcggcccaagcttagacactagagggtatataatggaagctcgacttccagcttggcaatccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

SEQ ID NO: 6 - DNA, 3563 bp, Artificial sequence
  Synthetic oligonucleotide
ggcctaactggccggtacctgagctcgctagcctcgaggatatcaagatctggcctcggcggccaagcttagacactagagggtatataatggaagctcgacttccagcttggcaatccggtactgttggtaaagccaccatggcttccaaggtgtacgaccccgagcaacgcaaacgcatgatcactgggcctcagtggtgggctcgctgcaagcaaatgaacgtgctggactccttcatcaactactatgattccgagaagcacgccgagaacgccgtgatttttctgcatggtaacgctgcctccagctacctgtggaggcacgtcgtgcctcacatcgagcccgtggctagatgcatcatccctgatctgatcggaatgggtaagtccggcaagagcgggaatggctcatatcgcctcctggatcactacaagtacctcaccgcttggttcgagctgctgaaccttccaaagaaaatcatctttgtgggccacgactggggggcttgtctggcctttcactactcctacgagcaccaagacaagatcaaggccatcgtccatgctgagagtgtcgtggacgtgatcgagtcctgggacgagtggcctgacatcgaggaggatatcgccctgatcaagagcgaagagggcgagaaaatggtgcttgagaataacttcttcgtcgagaccatgctcccaagcaagatcatgcggaaactggagcctgaggagttcgctgcctacctggagccattcaaggagaagggcgaggttagacggcctaccctctcctggcctcgcgagatccctctcgttaagggaggcaagcccgacgtcgtccagattgtccgcaactacaacgcctaccttcgggccagcgacgatctgcctaagatgttcatcgagtccgaccctgggttcttttccaacgctattgtcgagggagctaagaagttccctaacaccgagttcgtgaaggtgaagggcctccacttcagccaggaggacgctccagatgaaatgggtaagtacatcaagagcttcgtggagcgcgtgctgaagaacgagcagtaattctagagtcggggcggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaaaatcgataaggatccgtcgaccgatgcccttgagagccttcaacccagtcagctccttccggtgggcgcggggcatgactatcgtcgccgcacttatgactgtcttctttatcatgcaactcgtaggacaggtgccggcagcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagcggccgcaaatgctaaaccactgcagtggttaccagtgcttgatcagtgaggcaccgatctcagcgatctgcctatttcgttcgtccatagtggcctgactccccgtcgtgtagatcactacgattcgtgagggcttaccatcaggccccagcgcagcaatgatgccgcgagagccgcgttcaccggcccccgatttgtcagcaatgaaccagccagcagggagggccgagcgaagaagtggtcctgctactttgtccgcctccatccagtctatgagctgctgtcgtgatgctagagtaagaagttcgccagtgagtagtttccgaagagttgtggccattgctactggcatcgtggtatcacgctcgtcgttcggtatggcttcgttcaactctggttcccagcggtcaagccgggtcacatgatcacccatattatgaagaaatgcagtcagctccttagggcctccgatcgttgtcagaagtaagttggccgcggtgttgtcgctcatggtaatggcagcactacacaattctcttaccgtcatgccatccgtaagatgcttttccgtgaccggcgagtactcaaccaagtcgttttgtgagtagtgtatacggcgaccaagctgctcttgcccggcgtctatacgggacaacaccgcgccacatagcagtactttgaaagtgctcatcatcgggaatcgttcttcggggcggaaagactcaaggatcttgccgctattgagatccagttcgatatagcccactcttgcacccagttgatcttcagcatcttttactttcaccagcgtttcggggtgtgcaaaaacaggcaagcaaaatgccgcaaagaagggaatgagtgcgacacgaaaatgttggatgctcatactcgtcctttttcaatattattgaagcatttatcagggttactagtacgtctctcaaggataagtaagtaatattaaggtacgggaggtattggacaggccgcaataaaatatctttattttcattacatctgtgtgttggttttttgtgtgaatcgatagtactaacatacgctctccatcaaaacaaaacgaaacaaaacaaactagcaaaataggctgtccccagtgcaagtgcaggtgccagaacatttctct

(19) United States
(12) Patent Application Publication
(10) Pub. No.: US 2013/0045484 A1
(43) Pub. Date: Feb. 21, 2013

(54) METHODS AND KITS FOR DETERMINING THE TOXICITY OF AN AGENT

(71) Applicants: Jeffrey Kenneth Horton, Cardiff (GB); Peter James Tatnell, Cardiff (GB); Simon Laurence John Stubbs, Cardiff (GB)

(72) Inventors: Jeffrey Kenneth Horton, Cardiff (GB); Peter James Tatnell, Cardiff (GB); Simon Laurence John Stubbs, Cardiff (GB)

(73) Assignee: GE HEALTHCARE UK LIMITED, LITTLE CHALFONT (GB)

(21) Appl. No.: 13/695,042

(22) PCT Filed: Apr. 27, 2011
(86) PCT No.: PCT/EP2011/056685

§ 371 Date: Oct. 28, 2012

(30) Foreign Application Priority Data
Apr. 30, 2010 (GB) 1007197.5

(51) Int. Cl. C12Q 1/68; C12Q 1/66; G01N 21/75; C12Q 1/37; G01N 21/64; G01N 21/76; C12Q 1/18; C12Q 1/26

(52) U.S. Cl. 435/6.13; 435/32; 435/8; 435/25; 435/23

(57) ABSTRACT

The invention relates to methods and kits for determining the toxicity of an agent on a population of eukaryotic cells, particularly human cells. The cells may comprise a nucleic acid construct comprising a DNA damage induced response element operably linked to a sequence encoding a reporter gene. The multiplex methods and kits provide means for distinguishing between genotoxic and cytotoxic agents.

### TECHNICAL FIELD

The present invention relates to the field of toxicological testing in eukaryotic systems. In particular, the invention relates to testing physical and chemical agents for their toxicological effects on human cells.

### BACKGROUND

The identification of human toxicological hazard is an important step in the development of new chemicals such as foodstuffs, cosmetics, pharmaceuticals, agrochemicals and environmental chemicals to which humans may be exposed. This is particularly the case for pharmaceutical compounds which must undergo stringent toxicological testing before they receive approval for clinical use.

The current battery of tests for hazard identification, agreed upon by regulatory agencies and pharmaceutical companies from the United States, the European Union, and Japan, consists of a series of in vitro and in vivo toxicity assays (ICH, S2B, 1997, International Conference on Harmonisation of Technical Requirements for Registration of Pharmaceuticals for Human Use (ICH), available at ich.org; Cimino. M. 2006, Environ. Mol. Mutagen, 47, 362-390).

The European Union has enacted a new European Community Regulation on chemicals and their safe use (EC 1907/2006). It deals with the Registration, Evaluation and Authorization of Chemical (REACH) substances and entered into force on 1 Jun. 2007. The aim of REACH is to improve the protection of human health and the environment through the better and earlier identification of the intrinsic properties of chemical substances.

Under this Regulation, the requirements for toxicology assessment are very important. In addition, reproductive and developmental considerations may result in the restriction of many substances that are now in widespread use.

Although REACH has what appears to be stringent requirements for experimental animal studies, the Regulation discourages the use of vertebrate animals in testing, requiring laboratories to consider alternative methods. However, many of the established alternative animal methods are often problematic. Thus, there exists a need for the introduction and validation of new cell-based methods to predict and assess the potential toxic effect of chemicals on humans. Ideally, these methods should involve in vitro, laboratory testing methods that provide information on the molecular mechanism and nature of the agent's toxicity. Moreover, such methods should be relatively cheap to perform and amenable to high throughput screening in order to provide an indication of potential toxicity at an early stage of chemical evaluation.

### Cytotoxicity Assays

Cytotoxicity or necrosis is the process by which a physical agent, cell or chemical injures a cell leading to loss of viability and cell death. This process is often associated with loss of membrane integrity and mitochondrial swelling. There are many methods or assays for measuring eukaryotic cell death or cytotoxicity, most being based on plasma membrane permeability such as release of radio-isotopic labels (³H or ⁵¹Cr), dye exclusion (e.g. trypan blue) and cytoplasmic enzyme release (e.g. lactate dehydrogenase, adenylate kinase). Other methods rely on biological functions within the cell, such as measuring mitochondrial dehydrogenase activity (e.g. tetrazolium salt reduction), lysosomal integrity and activity (e.g. neutral red binding) and protein synthesis (e.g. sulforhodamine B fixation); see, for example, Xenometrix website (www.xenometrix.ch). Xenometrix offer multiple endpoint cytotoxicity test kits allowing the determination of up to four cytotoxicity parameters from the same cellular sample.

Cellular adenosine triphosphate (ATP) content is a marker of cellular energy status and viability. When cells undergo necrosis or apoptosis, their ATP levels decline rapidly. The measurement of ATP levels therefore provides another assay for measuring cytotoxicity.

In combination with luciferase, the addition of D-luciferin to cells generates light in the presence of ATP. The intensity of the luminescence is proportional to the intracellular ATP content (Crouch, S. et al., 1993, J. Immunol. Methods 160, 81-88). High throughput cytotoxicity assays based upon ATP production have therefore been developed (see, for example, www.celis.com),

### Oxidative Stress Assays

Oxidative stress is caused by the presence of any of a number of reactive oxygen species which the cell is unable to counterbalance. The result is damage to one or more biomolecules including DNA, RNA, proteins and lipids. Excess reactive oxygen species must be promptly eliminated from the cell by a variety of antioxidant defence mechanisms. Cellular antioxidant enzymes and other redox molecules serve to counterbalance these reactive species generated in the cell.

There are many markers of oxidative stress including:

DNA/RNA damage (e.g. 8-hydroxyguanosine, 8-hydroxydeoxyguanosine), reactive oxygen species and antioxidant enzymes (e.g. superoxide dismutase, catalase), lipid peroxidation (e.g. 4-Hydroxynonenal, Malondialdehyde, 8-iso-Prostaglandin F₂ alpha) and oxidative protein damage (e.g. 3-Nitrotyrosine). These markers can be used as assays to determine the oxidative stress of the cell.

A number of oxidative stress assays are available based upon measuring the levels of these biomarkers (e.g. Cellbiolabs at www.cellbiolabs.com)

### Apoptotic Assays

Apoptosis or programmed cell death is an active process that requires metabolic activity of the dying cell and involves signal transduction cascades. The caspases consist of a group of cysteine proteases which are activated during apoptosis. These unique proteases, which are synthesized as zymogens, are involved in the initiation and execution of apoptosis once activated by proteolytic cleavage. Mammalian caspases may be grouped by function: cytokine activation includes caspases 1, 4, 5, 13; apoptosis initiation includes caspases 2, 8, 9, 10; and apoptosis execution utilizes caspases 3, 6, 7. Caspase assays are based on the measurement of zymogen processing to an active enzyme and proteolytic activity. A number of commercial kits and reagents are available to assess apoptosis based on caspase function: PhiPhiLux® from Oncolmmunin, Inc for caspase-3 (website at www.philiphilux.com); CaspaLux® family of substrates for caspases 1, 6, 8 and 9 from Oncolmmunin, Inc (website at www.phiphilux.com); and the Caspase-3 Activity Assay (Roche at www.roche-applied-science.com). Assays that measure prelytic DNA fragmentation are well suited for the determination of apoptotic cell death because DNA cleavage is a hallmark of apoptosis. The DNA fragments may be assayed as either “ladders” (with the 180 bp multiples representing the “rungs” of the ladder) derived from cell populations or by ELISA quantification of histone-complexed DNA fragments (see, for example, Roche at www.roche-applied-science.com).

Another method for measuring apoptosis is by following the release of cytochrome C and apoptosis inducing factor from mitochondria into the cytoplasm. Apoptotic-specific alterations of mitochondria are more difficult to detect. The mitochondrial pathway begins with the permeabilization of the mitochondrial outer membrane by proapoptotic members of the Bcl-2 family, resulting in a release of cytochrome c and other toxic proteins from the intermembrane space into the cytosol. The release of these proteins is generally determined by immunocytochemistry or by Western blotting of cytosolic, mitochondrial and nuclear fractions.

Granzyme a and Granzyme b are serine proteases that are released by cytoplasmic granules within certain mammalian cells (such as cytotoxic T cells and natural killer cells). Their purpose is to induce apoptosis within virus-infected cells, thus destroying the infection. This process will normally involve other enzymes such as caspases.

Poly(ADP-ribose) polymerase (PARP) is an enzyme involved in a number of cellular processes involving mainly DNA repair and apoptosis. PARP also has the ability to directly induce apoptosis, via the generation of ADP-ribose polymer, which stimulates mitochondria to release apoptosis inducing factor. This mechanism is caspase-independent.

### Genotoxicity Testing

Genotoxic substances cause damage to the genetic material of cells and are potentially mutagenic or carcinogenic, Direct DNA damage is induced by a variety of agents such as UV light, X-rays, free radicals and alkylating agents. DNA damage can also be caused indirectly either by pro-mutagens or by agents that affect enzymes and proteins which interact with DNA. Therefore genotoxic substances have the ability to cause damage to the genetic material of cells and are therefore potentially mutagenic or carcinogenic. Although a number of assays for measuring mutation rates in vitro and in vivo have been developed, improved methods are needed for the understanding of the risk prediction and safety of chemical and physical agents.

Genotoxicity testing is performed to ensure safety during clinical trials and during the treatment of general patient populations (Cimino M. 2006, Environ. Mutagen, 47, 362-390).

Various methods, such as i) the Ames test, ii) the in vitro micronucleus test and iii) the mouse lymphoma assay, are used to assess the toxicity of an agent, however all of these are unsatisfactory for a number of reasons. For instance, prolonged incubation times are often required; this is limiting especially when it is desirable to obtain genotoxic data in a shorter time-frame. Furthermore, many known methods of detecting DNA damage (including the Ames Test), measure DNA damage at an undefined endpoint e.g. a gene knock-out mutation. Subtle effects such as reduced activity or gene expression are therefore not detected by these systems. An alternative GFP-based mutation assay using the GADD45α promoter has been described (Hastwell et al., 2006, Mutat. Research, 607, 160-175). However, this method is technically unreliable and prone to difficulties. Ohno et al., 2005, Mutat. Research, 588, 47-57), described an alternative genotoxicity test system based upon a single measurable output using a p53 response element from the p53R2 promoter in a luciferase gene reporter assay.

Hastwell et al., (2006, Mutat. Research, 607, 160-175) described a gene reporter system based on the human GADD45α gene. The recognition of GADD45α as a biomarker for genomic stress and damage has made it possible to engineer a reporter system in which the GADD45α promoter is fused to the cDNA encoding GFP. This has allowed the development of a 96-well microplate assay (GreenScreen HC, www.gentronix.co.uk) to identify genotoxins. However this assay has a number of reported limitations (Olaharski et al., 2009, Mutat. Research, 672, 10-16).

Ohno et al. (2008, Mutat. Research 656, 27-35; 2005, Mutat. Research 588, 47-57) reported the construction of an alternative genotoxic luciferase gene reporter assay based on three tandem repeat sequences of the p53 response element from the p53R2 promoter. One of the p53 target genes activated by genotoxic compounds is p53R2. The p53R2 gene product supplies nucleotides to repair damaged DNA (Tanaka et al., 2000, Nature, 404, 42-49; Xue et al., 2003, Cancer Res. 63, 980-986).

Expression of p53R2 is known to be activated by γ-rays, UV light and genotoxic compounds in a p53-dependent manner (Tanaka et al., 2000, Nature, 404 42-49).

### Technical Problem

Existing cellular assays generate limited data for determining the toxicity of an agent on eukaryotic cells. Several separate tests are required to develop an understanding of the mechanisms underlying the agent's toxicity to the cell. There is therefore a need for multiplex cellular assays which provide a range of information which can be used to generate a toxicological profile for any agent. Ideally such assays should be simple, cheap and amenable to scale-up for high throughput screening and high content screening/analysis.

### DEFINITIONS

The term “agent” as used herein describes a physical stimulus (e.g. light, heat, radiation), chemical treatment or biological cell (e.g. cytotoxic T cell) which may be toxic to a eukaryotic cell. The chemical treatment may comprise a single compound or a mixture of compounds.

As disclosed herein, the term “multiplex assay” or “multiplex method” relates to or is a method of measurement or communication of information or signals from two or more messages from the same source (an example of a multiplex assay is described by Ugozzoli, et al. 2002 Anal. Biochem., 307, 47-53). Multiplex assays are distinguished from procedures that measure single analytes or single biomarkers.

The term “cytotoxicity” as used herein describes a process by which an agent injures an eukaryotic cell leading to loss of viability and cell death.

The term “oxidative stress” as used herein relates to an imbalance in reactive oxygen species present within an eukaryotic cell which the cell is unable to counterbalance.

The term “apoptosis” as used herein relates to an active process that requires metabolic activity of the dying cell and involves signal transduction cascades. “Apoptotic activity” describes the process by which an agent elicits programmed cell death in an eukaryotic cell.

The term “genotoxicity” as used herein describes a deleterious action of an agent on a cell's genetic material affecting its integrity. Genotoxic agents are known to be potentially mutagenic or carcinogenic, specifically those capable of causing genetic mutation and of contributing to the development of tumours.

The term “mutagen” as used herein describes a physical or chemical agent that changes the genetic material of an organism and thus increases the frequency of mutations above the natural background level. As many mutations cause cancer, mutagens are typically also carcinogens.

The term, “high-content screening”, as used herein, is a drug discovery method that uses living cells as the test tube for molecular discovery. It describes the use of spatially or temporally resolved methods to discover more from an individual experiment than one single experiment with one output alone. It uses a combination of cell biology, with molecular tools, typically with automated high resolution microscopy and robotic handling (Giuliano et al., 1997, J Biomol Screen., 2, 249-259).

A “biomarker”, or “biological marker”, as used herein is a cellular substance used as an indicator of a biological state. It is a characteristic that is objectively measured and evaluated as an indicator of normal biological processes, pathogenic processes, toxicity or pharmacologic responses to a therapeutic intervention. Examples of biomarkers are ATP, Caspase-3 and Superoxide Dismutase.

The term “cell growth” or “cell proliferation” as used herein describes cell division. The term refers to growth of cell populations, where one cell grows and undergoes cell division to produce daughter cells.

### SUMMARY OF THE INVENTION

According to a first aspect of the present invention, there is provided a multiplex method for determining the toxicity of an agent on a population of eukaryotic cells, wherein the population of cells optionally comprises a nucleic acid construct comprising a DNA damage induced response element operably linked to a sequence encoding a reporter gene, the method comprising
i) contacting a first population of eukaryotic cells in a test vessel with an agent to produce a test sample and contacting a second population of the cells in a control vessel with a control treatment to produce a control sample;
ii) lysing the cells in both the test sample and the control sample
iii) quantifying the levels of ATP present in both the test sample and the control sample, wherein a difference in the levels of ATP between the samples is indicative of the cytotoxicity of the agent; and
iv) measuring the activity of an endogenous enzyme or reporter protein in both the test sample and the control sample, wherein a difference in the activities between the samples is indicative of the oxidative stress, apoptotic activity or genotoxicity induced by the agent;
   wherein steps i) to iv) are carried out on the same test sample in the same test vessel and on the same control sample in the same control vessel.

In one aspect, a luciferase enzyme is added to quantify the levels of ATP in step iii).

In another aspect, the activity of an oxidative enzyme is measured in step iv) as indicative of the oxidative stress induced by the agent. The oxidative enzyme may be, for example, superoxide dismutase.

In a further aspect, the activity of a proteolytic enzyme is measured in step iv) as indicative of the apoptotic activity of the agent. Preferably, the proteolytic enzyme is selected from the group consisting of caspase 1, caspase 3, caspase 7, caspase 8, caspase 9, granzyme a and granzyme b. More preferably, the caspase enzyme is caspase-3.

In yet a further aspect, the test vessel and the control vessel are wells in a microwell plate or a multiwall plate. The test vessel and control vessel may be wells or retainers in other forms of receptacles or containers which are suitable for retaining and/or maintaining cell populations.

In one aspect, the population of cells comprises a nucleic acid construct comprising a DNA damage induced response element operably linked to a sequence encoding a reporter gene, the method comprising the step of measuring the activity of the reporter gene protein product in both the test sample and the control sample, wherein a difference in the activity between the samples is indicative of the genotoxicity of the agent. Preferably the activity of the reporter gene is measured prior to lysing step ii) if the reporter gene product is a fluorescent protein. A range of fluorescent proteins are known, such as green fluorescent protein, cyan fluorescent protein, red fluorescent protein and blue fluorescent protein which produce an optical signal that can be measured using an imaging device, such as the IN Cell Analyzer 2000 from GE Healthcare.

In another aspect, the method additionally comprises the step of adding a substrate of the reporter gene protein product to both the test sample and the control sample under conditions to permit expression of the reporter gene prior to measuring the activity in both the test sample and the control sample. Preferably, this step is carried out after step i) and prior to lysing step ii).

In a further aspect, the DNA damage induced response element is located within a group of promoters selected from the group consisting of p53R2, GADD45α, mrt-2, hus-1, rad-5, cep-1, egl-1, ape-1, abl-1, brc-1, brd-1, pme-5, kin-2-, hpr-9, hpr-17, chk-1 and chk-2. Preferably, the DNA damage induced response element is located within the p53R2 promoter.

In one aspect, the nucleic acid construct additionally comprises a promoter sequence. Preferably, the promoter sequence is selected from the group of promoters consisting of p53R2, GADD45α, mrt-2, hus-1, rad-5, cep-1, egl-1, ape-1, abl-1, brc-1, brd-1, pme-5, kin-2-, hpr-9, hpr-17, chk-1 and chk-2. More preferably, the promoter is the p53R2 promoter.

In another aspect, the reporter gene is selected from the group consisting of a fluorescent protein, a luciferase, β-lactamase, a dihydrofolate reductase, a β-glucuronidase, β-galactosidase, a chloramphenicol acetyltransferase, a ubiquitinase, an alkaline phosphatase, a tryptophan synthase reporter gene and a nitro reductase reporter gene. Preferably, the reporter gene is a luciferase gene. More preferably, the reporter gene is a Renilla luciferase gene.

In a further aspect, the DNA damage induced response element is derived from the p53R2 promoter, the reporter gene is a Renilla luciferase, and a firefly luciferase enzyme is added to quantify the levels of ATP in step iii).

In one aspect the eukaryotic cell is a nematode cell, for example Caenorhabditis elegans. C. elegans provides many advantages for the study of DNA surveillance and repair in a multicellular organism. Several genes have been identified by mutagenesis and RNA interference that affect DNA damage checkpoint and repair functions. Many of these DNA damage response genes also play essential roles in DNA replication, cell cycle control, development, meiosis and mitosis and may prove useful in a genotoxicity assay system (O'Neil & Rose, 2006: In, Blumenthal, T. (Ed) The WormBook, www. WormBook.org)

Preferably, the eukaryotic cell is a mammalian cell. More preferably, the mammalian cell is a human cell. There are many human cell lines which can be used in the first aspect of the invention, such as the human embryo kidney (HEK) 293 cell line.

In one aspect, the agent is a form of electromagnetic radiation. Different forms of electromagnetic radiation are known to be toxic to eukaryotic cells, particularly high energy forms of radiation such as gamma radiation, x-rays and ultra violet radiation.

In another aspect, the agent is an organic or inorganic compound. Examples of organic compounds include, for example, proteins, peptides, nucleic acids, carbohydrates, lipids, and synthetic compounds which have been designed as potential drugs, insecticides, herbicides, fungicides, antivirals and antibiotics.

In a further aspect, the method additionally comprises correlating the cytotoxicity of the agent with any or all of the genotoxicity, oxidative stress or apoptotic inducing activities of the agent.

According to a second aspect of the present invention, there is provided a method of profiling the toxicity of an agent comprising the method as hereinbefore described.

In a third aspect of the present invention, there is provided a kit comprising a population of cells and instructions for carrying out the method as hereinbefore described.

According to a fourth aspect of the present invention, there is provided a kit comprising a population of cells comprising a nucleic acid construct comprising a DNA damage induced response element operably linked to a sequence encoding a reporter gene and instructions for carrying out the method as hereinbefore described.

In a fifth aspect of the present invention, there is provided a kit comprising a vector comprising a nucleic acid construct comprising a DNA damage induced response element operably linked to a sequence encoding a reporter gene and instructions for carrying out the method as hereinbefore described.

According to a sixth aspect of the present invention, there is provided a use of a method or a kit as hereinbefore described for drug development, toxicological screening or toxicological profiling.

### BRIEF DESCRIPTION OF THE DRAWINGS

In description of the method of the invention reference is made to the following figures:

FIG. 1: Diagram of hybridised oligonucleotides p53R2RE For and Rev;

FIG. 2: Vector Map of pGL4.70 [hRLuc];

FIG. 3: Vector Map of p53R2 (RE) pGL4.70 [hRLuc];

FIG. 4: Vector Map of p53R2 (RE) pGL4.70 MinP [hRLuc];

FIG. 5: Vector Map of pGL4.70 MinP[hRLuc];

FIG. 6: Firefly luciferase response based on 20-45000 HEK 293 cells per well;

FIG. 7: Firefly luciferase response based on 20-2800 HEK 293 cells per well;

FIG. 8: Firefly luciferase response based on 2-1000 nM ATP;

FIG. 9: Firefly luciferase response based on 2-62.5 nM ATP;

FIG. 10 shows results from (a) the p53R2 Renilla Luciferase Reporter Gene Assay. HEK 293 cells were transfected with 5 μl from a 8:2 transfection complex p53R2 (RE) pGL4.70 MinP [hRLuc]) and treated with known amounts of mannitol (1 mg/ml), phenformin hydrochloride (‘phenformin’; 250 μg/ml), methylmethane sulfonate (‘MMS’, 50 μg/ml) or doxorubicin hydrochloride (‘doxorubicin’, 1 μg/ml); (b) control (cells treated as in (a) above, but with pGL4.70 MinP [hRLuc]); (c) p53R2 Renilla Luciferase Reporter Gene Assay. HEK 293 cells were transfected with 5 μl from a 8:2 transfection complex (8 μl of transfection reagent and 2 μg p53R2 (RE) pGL4.70 [hRLuc]) and treated with known amounts of mannitol, phenformin, methylmethane sulfonate or doxorubicin hydrochloride prior to p53R2 induction; (d) control (cells treated as in (c) above, but with pGL4.70 [hRLuc]); (e) ATP Bioluminescent ATP cell viability assay (ATP bioassay) results. Results are shown in RLU and data as a mean±1 SD (n=3). P<0.05 were significant results as compared with the control (cells only, without treatment with compound); N.S. indicates no statistically significant difference.

FIG. 11 depicts results from (a) the p53R2 Renilla Luciferase Reporter Gene Assay. HEK 293 cells were transfected with 5 μl from a 7:2 transfection complex (7 μl of transfection reagent and 2 μg p53R2 (RE) pGL4.70 MinP [hRLuc]) and treated with known amounts of mannitol (1 mg/ml), phenformin (250 μg/ml), methylmethane sulfonate (50 μg/ml) or doxorubicin hydrochloride (1 μg/ml); (b) control (cells treated as in (a) above, but with pGL4.70 MinP [hRLuc]); (c) p53R2 Renilla Luciferase Reporter Gene Assay. HEK 293 cells were transfected with 5 μl from a 7:2 transfection complex (7 μl of transfection reagent and 2 μg p53R2 (RE) pGL4.70 [hRLuc]) and treated with known amounts of mannitol, phenformin, methylmethane sulfonate or doxorubicin hydrochloride; (d) control (cells treated as in (c) above, but with pGL4.70 [hRLuc]); (e) ATP Bioluminescent ATP cell viability assay results. The results are shown in RLU and are the mean±1 SD (n=3). P<0.05 were significant results as compared with the control (cells only, without treatment with compound) with N.S. indicating no statistically significant difference.

FIG. 12: Dose-response curves for p53R2 Renilla luciferase reporter gene assay following treatment with: doxorubicin; methylmethane sulfonate; mannitol or phenformin;

FIG. 13: Dose-response curves for firefly luciferase bioluminescent ATP cell viability assay following treatment with: doxorubicin; methylmethane sulfonate; mannitol or phenformin;

FIG. 14: Dose-response curves for superoxide dismutase (SOD) following treatment with: doxorubicin; methylmethane sulfonate; mannitol or phenformin (results are shown as absorbance units at 450 nm);

FIG. 15: Dose-response curves for superoxide dismutase (SOD) following treatment with: doxorubicin; methylmethane sulfonate; mannitol or phenformin (results are shown as percentage inhibition relative to untreated control cells);

FIG. 16: Dose-response curves for Caspase-3 apoptosis assay following treatment with: doxorubicin; methylmethane sulfonate; mannitol or phenformin (results are shown as absorbance units at 405 nm);

FIG. 17: Genotoxicity Index as measured as a function of ATP measurements and p53R2 Renilla activity; and

FIG. 18: (a) Cellular stress Index as measured as a function of ATP measurement and superoxide dismutase activity; and b) Apoptotic Index as measured as a function of ATP measurement and caspase-3 activity.

### SEQUENCE LISTING

SEQ ID No: 1 is nucleotide sequence of p53R2 For;

SEQ ID No: 2 is the nucleotide sequence of p53R2Rev;

SEQ ID No. 3 is the nucleotide sequence of pGL4.70 [hRLuc];

SEQ ID No. 4 is the nucleotide sequence of p53R2(RE) pGL4.70[hRLuc]

SEQ ID No. 5 is the nucleotide sequence of p53R2(RE) pGL4.70 MinP [hRLuc]; and

SEQ ID No. 6 is the nucleotide sequence of pGL4.70 MinP[hRLuc];

### DETAILED DESCRIPTION

### Molecular Biology

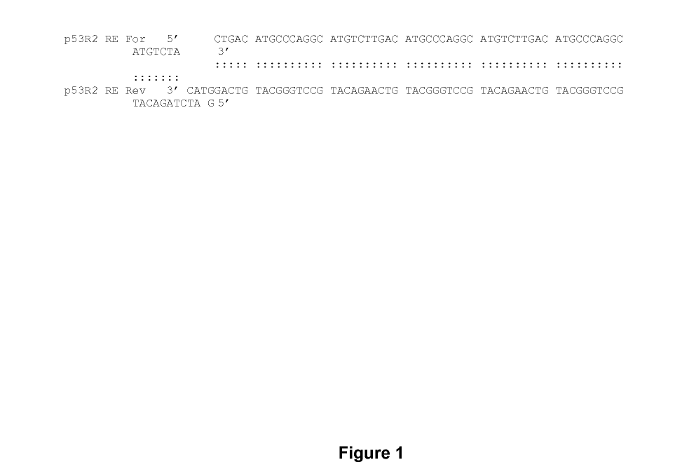

Complementary oligonucleotides encoding the consensus DNA binding sequences for p53 (5′-Pu Pu Pu C (A/T) (A/T) G Py Py Py-3′) were designed according to the p53 binding sequence located in intron 1 of the human p53R2 gene (Ohno, K. et al., 2005 Mut. Res., 588, 47-57). These oligonucleotides designated p53R2 (RE) For (SEQ ID No: 1) and Rev (SEQ ID NO: 2) were 62 and 70 base pairs respectively and when hybridized together generated a double stranded DNA molecule that possessed at its 5′- and 3′-prime regions over-hanging sequences that were compatible with the restriction enzymes Kpnl and BglII (FIG. 1). Both oligonucleotides were synthesized by Sigma Life Science in a non-phosphorylated format and purified using polyacrylamide gel electrophoresis (PAGE).

Purified oligonucleotides were resuspended in molecular biology grade water (Sigma, catalogue no. W502) at 100 μM and hybridized together by dispensing 20 μg (˜1 nmole) of each in STE buffer (Sigma, catalogue no. 85810) at a final concentration of 0.5× (w/v). This was performed in a volume of 40 μl. The reaction mixture was heated to 95° C. for 5 min and allowed to cool slowly to room temperature. A brief centrifugation was performed to pool the reaction mixture at the bottom of the tube.

In order to confirm hybridisation and hence the generation of a double stranded DNA product an aliquot (2 μl) containing ˜2 μg of the annealed oligonucleotides were analysed by 2% agarose gel electrophoresis. This sample was compared to comparable amounts of un-hybridised oligonucleotides.

The remaining double stranded DNA product was purified using the Illustra™ GFX PCR Band and Gel band purification kit (GE Healthcare catalogue no. 28-9034-70) after 2% agarose gel electrophoresis. This product was designated p53R2 (RE) and possessed 5′- and 3′-prime over-hanging ends that were compatible with the restriction enzymes Kpnl and BglII respectively.

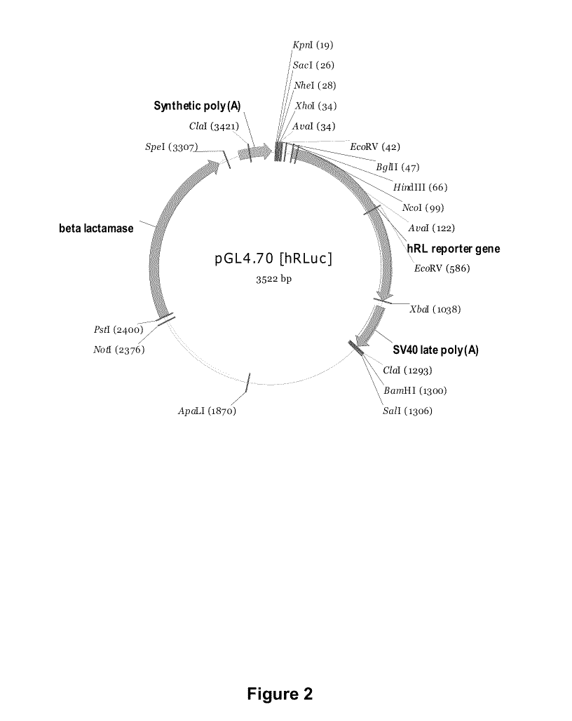

The vector pGL4.70 [hRLuc] (Promega, catalogue no. E6881, see FIG. 2 and Sequence ID NO: 3) contains the cDNA encoding the Renilla luciferase. This vector was digested with Kpnl and BglII (NEB catalogue nos. R0142S and R0144S respectively) and the appropriate product (˜3.6 kb) was purified using 1% agarose gel electrophoresis and the illustra GFX PCR Band and Gel band purification kit. The p53R2 (RE) product was sub-cloned into the pGL4.70 [hRLuc] using T4 DNA ligase (NEB catalogue no. MO201S).

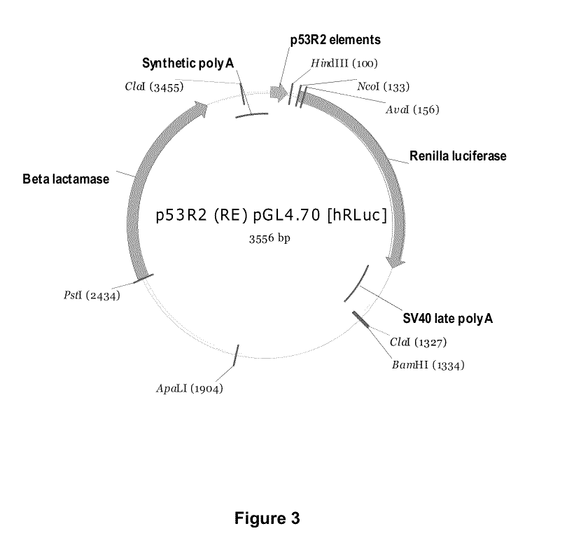

Successful sub-cloning was confirmed by diagnostic restriction enzyme digests and the sequencing of the relevant portion of the recombinant DNA molecule. The resultant sub-clone was designated p53R2 (RE) pGL4.70 [hRLuc] see FIG. 3 and Sequence ID NO: 4.

Similar methods and diagnostic analyses as those described above were performed to characterise and authenticate the subsequent clonings (described below).

The DNA construct described above lacks a minimal promoter. Natural promoters contain specific DNA sequences and response elements which provide a binding site for RNA polymerase and for transcription factors that recruit RNA polymerase. Promoters represent critical elements that can work in concert with other regulatory regions (enhancers, silencers, boundary elements/insulators) to direct the level of transcription of a given gene.

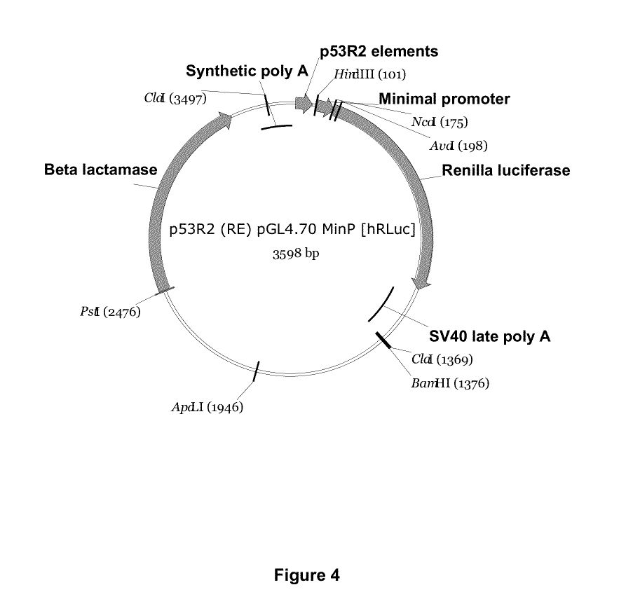

To facilitate transcription of the Renilla luciferase gene, the minimal promoter from the vector pGL4.23 [Luc2/minP] (Promega catalogue no. E8411) was excised on an 80 base pair NcoI and HindIII fragment (NEB catalogue nos. R0193S and R01404S respectively) and sub-cloned into the equivalent site present in the vector p53R2 (RE) pGL4.70 [hRLuc] described above. The NcoI and HindIII sites are positioned downstream in a 3′-prime location relative to the sequence encoding the p53R2 (RE) product. The resultant recombinant DNA molecule was designated p53R2 (RE) pGL4.70 MinP [hRLuc] (see FIG. 4 and SEQ ID NO: 5).

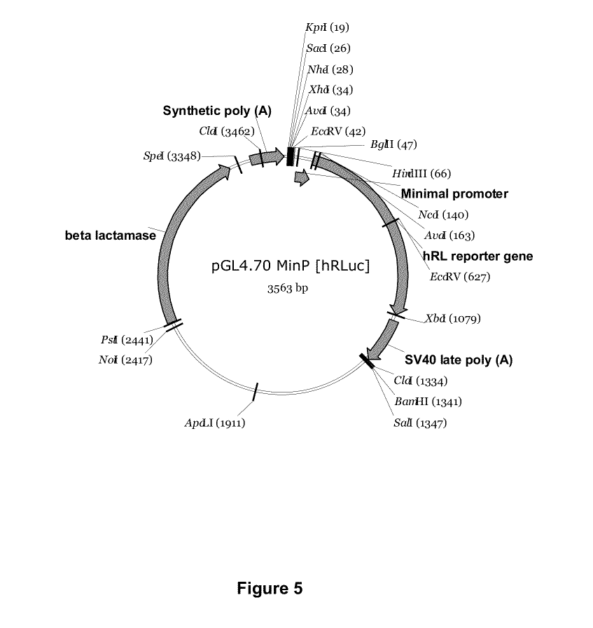

To generate a DNA molecule that could function as an appropriate control for subsequent transfection experiments and genotoxic cell-based assays a similar sub-cloning was performed in which the 80 base pair MinP NcoI and HindIII fragment from the vector pGL4.23 [Luc2/minP] was ligated into equivalent sites in the pGL4.70 [hRLuc] vector. The resultant construct was designated pGL4.70 minP [hRLuc] (see FIG. 5 and SEQ ID NO: 6). The pGL4.70 minP [hRLuc] DNA molecule generated was used to assess the impact of the p53R2 (RE) sequence in the p53R2 (RE) pGL4.70 MinP [hRLuc] construct in the presence of genotoxic and cytotoxic compounds.

### Reagents

HEK 293 (human embryo kidney) cells were obtained from the Health Protection Agency Culture Collection, Porton Down, UK. The ViviRen Live Cell Renilla Luciferase Substrate was purchased from Promega (see www.promega.com). The Cell-Titer Glo Luciferase ATP Quantification Assay Kit was supplied from Promega.

The Superoxide Dismutase and Caspase 3 assay kits used were those available from Sigma (www.sigmaaldrich.com).

All other chemicals used were the highest purity grade available and were sourced from Sigma.

### Cell Culture

HEK 293 cells were grown in Eagle's minimum essential medium (EMEM) supplemented with 10% (v/v) foetal calf serum, 4 mM L-glutamine, 100 U/ml penicillin, and 100 μg/ml streptomycin (all obtained from Life Technologies). Transfection experiments were carried out, in culture media as above, in the absence of antibiotics. Cells were routinely grown at 37° C. in a 95% water-saturated atmosphere containing 5% CO₂. Cell numbers were estimated using a haemocytometer.

### Transfections

Constructs were transiently transfected into human HEK 293 cells using a lipid-based commercially available reagent (FuGENE HD Transfection Reagent, Roche Applied Science). Transfections were carried out following the manufacturer's instructions. Briefly, freshly passaged cells (20,000/well) were dispensed into Greiner Bio-One tissue-culture treated μ-clear (clear base), white, sterile, 96-well cluster plates and incubated overnight (37° C., 95% humidity, 5% CO₂) in antibiotic-free media. Cells were transfected (100 ng DNA/well; 5 μl transfection complex/well) with pGL4.70 [hRLuc], p53R2 (RE) pGL4.70 [hRLuc], pGL4.70 MinP [hRLuc], or p53R2 (RE) pGL4.70 MinP [hRLuc] for 24 h, in antibiotic-free media. Several FuGENE HD Transfection Reagent: DNA ratios (3:2-8:2 in the final complex) were evaluated in order to determine the optimal level of transient transfection.

p53R2 Renilla Luciferase Reporter Gene Assay

Following transfection, test compounds (mannitol, doxorubicin hydrochloride (DOX), methylmethane sulfonate (MMS) and phenformin hydrochloride), or solvent (0.2% v/v DMSO), were incubated (37° C., 95% humidity, 5% CO₂ in antibiotic-free media) for up to 48 h. Renilla luciferase activity was measured using the ViviRen Luciferase Live Cell Substrate following the Manufacturer's instructions. Briefly, the ViviRen substrate (60 mM; 20 μl) was diluted 1:50 with complete EMEM at room temperature. 10 μl was added to each culture well (to give a final concentration of 60 μM in each well containing cells to be tested). Microtitre plates were incubated for two minutes at room temperature before measuring luminescence on a Tecan Ultra (Tecan Corporation) in luminescence mode, integrating for 100 ms. Alternatively luminescence can be measured on a LEADseeker multi-modality instrument in luminescence mode (GE Healthcare). Doxorubicin hydrochloride and methylmethane sulfonate (MMS) are reported to be genotoxic compounds, phenformin hydrochloride to be cytotoxic and mannitol to be non-toxic.

### Firefly Luciferase Bioluminescent ATP Assay Measurement

The assay was carried out in 96-well cluster plates on the same cell containing sample(s) as the p53R2 Renilla Luciferase Reporter Gene Assay. Traditionally bioluminescent ATP quantification methods use firefly luciferase to quantify ATP in the presence of inorganic magnesium ions and the substrate luciferin. The ATP assay described here uses a recombinant luciferase from a gene isolated from the Pennsylvania Firefly (Photuris pennsylvania).

Following Renilla luciferase gene reporter activity quantification, firefly luciferase activity was measured in each well using the Cell-Titer Glo Luciferase ATP Quantification Assay Kit in accordance with the kit manufacturer's instructions. Briefly, the enzyme substrate was equilibrated to room temperature and reconstituted with the assay buffer. 100 μl of this reagent was added to cells which quenched the Renilla luciferase reaction and liberated the cellular contents by cellular lysis. The contents of each plate were incubated for 2 mins on an orbital shaker, before measurement of bioluminescence on a Tecan Ultra (Tecan Corporation) in luminescence mode, integrating for 100 ms. Alternatively bioluminescence can be measured on a LEADseeker multi-modality instrument in luminescence mode (GE Healthcare).

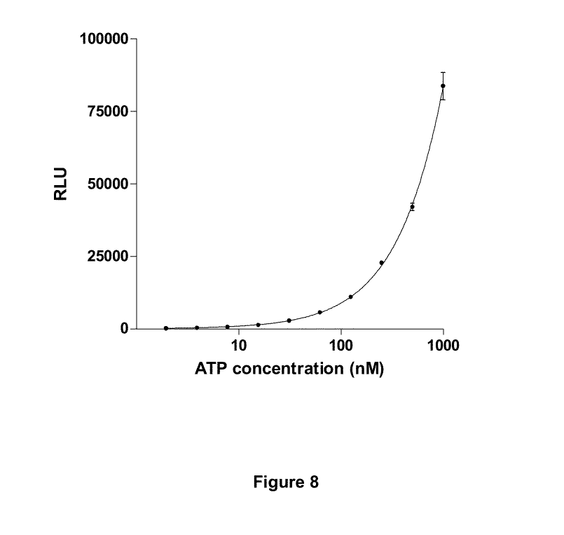

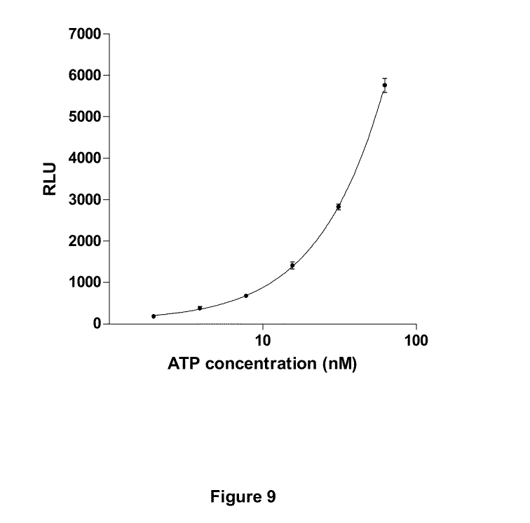

For genotoxicity testing for potential mutagens active in cell cultures, this ATP assay provides a simple, accurate and convenient method for determining the number of viable cells and measurement of cytotoxicity in culture. It is based on the quantitative measurement of ATP which indicates the absolute number of metabolically active cells. Alternatively the method can be compared with dose-response curves prepared using a range of concentrations of standard amounts of ATP prepared in complete cell culture media, thus allowing for simple overall standardisation of the technique, from well to well, plate to plate and assay to assay, improving total assay reproducibility. The method described herein is sensitive to at least 100 cells in culture and less than 10 nM ATP (see, for example, FIGS. 8 and 9).

### Superoxide Dismutase (SOD) Measurement

The assay was conducted using the same cell containing sample(s) as the p53R2 Renilla Luciferase Reporter Gene and the Firefly Luciferase Bioluminescent ATP assays. The assays were performed in 96-well cluster plates.

SOD serves a key antioxidant role in all cells and is indicative of oxidative stress which is caused by an imbalance between the production of reactive oxygen and the cells' ability to readily detoxify the reactive intermediates or easily repair the resulting damage. Disturbances in oxidative stress can cause toxic effects through the production of peroxides and free radicals that damage all components of the cell, including proteins, lipids, and nucleic acids. Thus, the SOD assay may provide useful information on the mechanism of cellular toxicity.

On completion of ATP quantification, SOD was measured on each cellular sample following the SOD assay kit manufacturer's instructions. Briefly, the assay method is based on a water-soluble tetrazolium salt, WST-1 (2-(4-Iodophenyl)-3-(4-nitrophenyl)-5-(2,4-disulfophenyl)-2H-tetrazolium, monosodium salt) that produces a colourimetric formazan dye upon reduction with a superoxide anion which can be measured spectrophotometrically. The rate of the reduction with the superoxide anion is linearly related to the inhibition of xanthine oxidase (XO) activity. Thus, cellular samples (20 μl) were added to a reaction mixture (220 μl) containing WST-1 and XO and incubated for 20 mins at 37° C. Absorbance was measured at 450 nm using a 96-well microtitre plate reader.

### Caspase-3 Assay

The assay was carried out using the same cell containing sample(s) as the p53R2 Renilla Luciferase Reporter Gene, the Firefly Luciferase Bioluminescent ATP and the superoxide assays. Once again, all of the assays were performed in 96-well cluster plates. Activation of caspase-3 plays a central role in the execution-phase of apoptosis. Thus, the caspase-3 assay will provide useful information on the mechanism of cellular toxicity. Following SOD measurement, caspase-3 was measured on each cellular sample in accordance with the Caspase-3 Assay Kit manufacturer's instructions. Briefly, the colorimetric assay is based on the hydrolysis of the peptide substrate acetyl-Asp-Glu-Val-Asp p-nitroanilide (Ac-DEVD-pNA) by caspase-3, resulting in the release of the p-nitroaniline (pNA) moiety which can be measured at 405 nm.

For the assay, cellular sample lysates (5 μl) were added to a reaction mixture containing caspase-3 substrate (Ac-DEVD-pNA, 95 μl) and samples were incubated for 2 h at 37° C. Absorbance was measured at 405 nm using a 96-well microtitre plate reader.

### Data Processing

Non-parametric statistics were used to determine the differences between control and experimental results. P values of less than 0.05 were taken as statistically significant.

### Discussion of Results

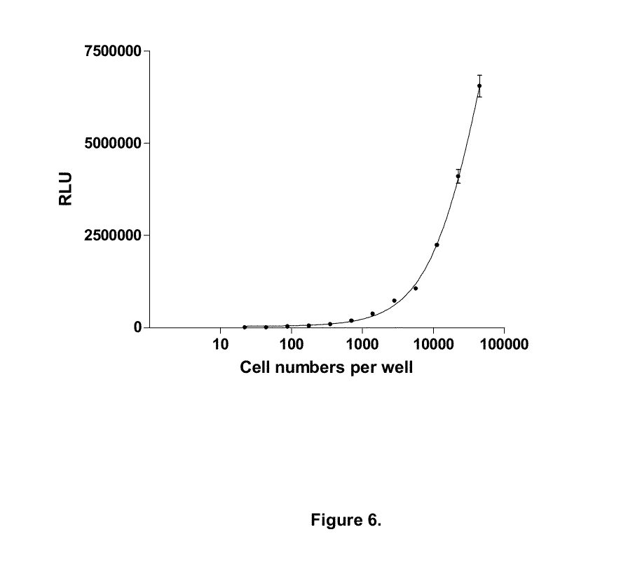

FIG. 6 illustrates firefly luciferase measurement of ATP from cultured HEK 293 cells (20-45000 cells/well); the results are given as the mean±1SD from three different measurements and are shown in Relative Light Units (RLU). This assay provides an index of cell numbers and viability, when ATP is measured from actively metabolising cells. The assay method gives results as an index of live cell numbers.

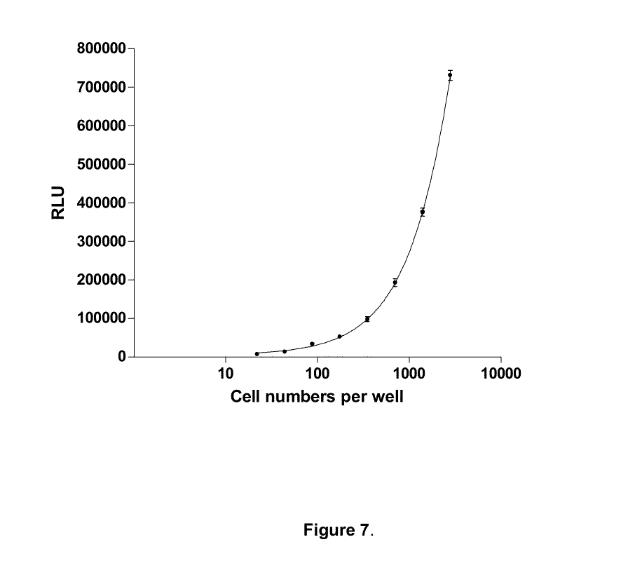

Similar data are presented in FIG. 7 which depicts firefly luciferase measurement of ATP from cultured HEK 293 cells (20-2800 cells/well). The results are shown as the mean±1 SD from three different measurements in RLU. This assay provides an index of cell numbers and viability, when ATP is measured from actively metabolising cells. The assay is clearly sensitive to less than 100 cells/well and gives results as an index of live cell numbers.

FIG. 8 shows firefly luciferase measurement from a range of concentrations (2-1000 nM) of known amounts of crystalline ATP (Sigma; Catalog code FL-AAS); results are the mean±1SD from three different measurements and are shown in RLU. These assay data provide a means for standardising the cell viability assay technique from batch to batch and assay to assay, thereby improving the accuracy and reproducibility of the technique.

Similar data are presented in FIG. 9 which shows firefly luciferase measurement from a lower range of concentrations (2-62.5 nM) of known amounts of crystalline ATP (Sigma; Catalog code FL-AAS), the results are the mean±1SD from three different measurements and are shown in RLU. These data provide a means for standardising the cell viability assay technique from batch to batch and assay to assay, thus improving the accuracy and reproducibility of the technique. The assay is clearly sensitive to less than 10 nM ATP.

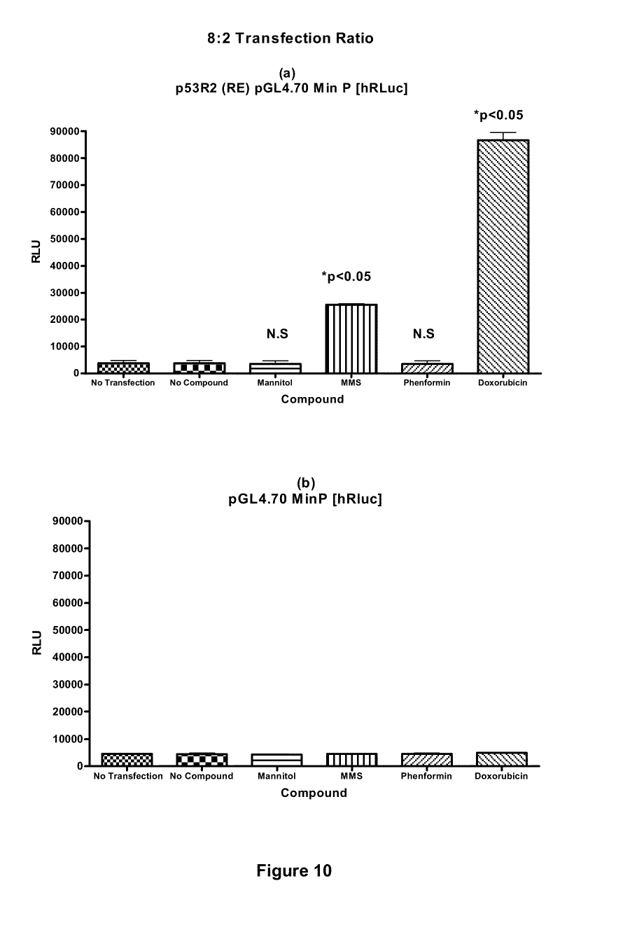

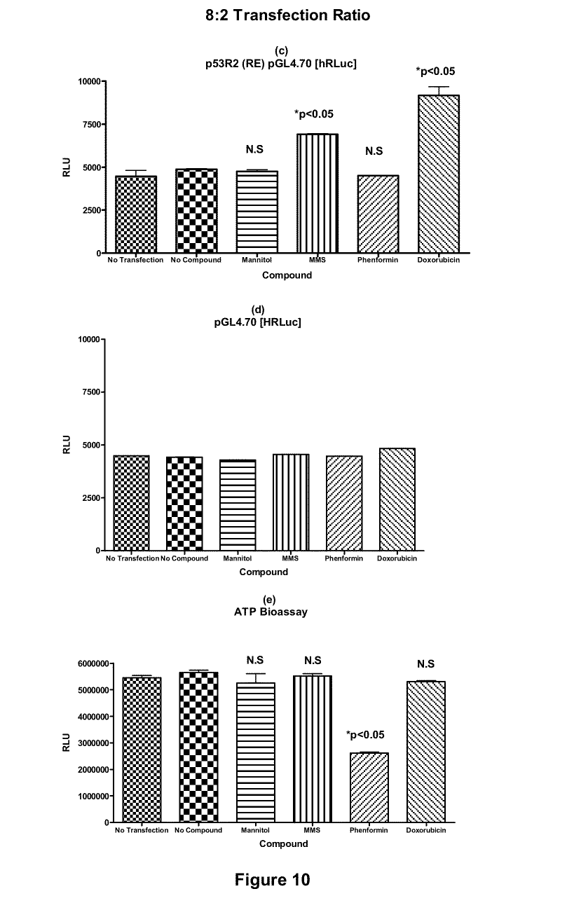

FIG. 10 shows results from (a) the p53R2 Renilla Luciferase Reporter Gene Assay. Cells were transfected with 5 μl from a 8:2 transfection complex (8 μl of transfection reagent and 2 μg p53R2 (RE) pGL4.70 MinP [hRLuc]). Transfected HEK 293 cells were treated with known amounts of mannitol (1 mg/ml), phenformin (250 μg/ml), methylmethane sulfonate (50 μg/ml) or doxorubicin hydrochloride (1 μg/ml); (b) control (cells treated as in (a) above, but with pGL4.70 MinP [hRLuc]); (c) p53R2 Renilla Luciferase Reporter Gene Assay. Cells were transfected with 5 μl from a 8:2 transfection complex (8 μl of transfection reagent and 2 μg p53R2 (RE) pGL4.70 [hRLuc]). Transfected HEK 293 cells were treated with known amounts of mannitol, phenformin, methylmethane sulfonate or doxorubicin hydrochloride prior to p53R2 induction; (d) control (cells treated as in (c) above, but with pGL4.70 [hRLuc]); (e) ATP Bioluminescent ATP cell viability assay results. Results are shown in RLU and data as a mean±1SD (n=3). P<0.05 were significant results as compared with the control (cells only, without treatment with compound); N.S. indicates no statistically significant difference.

The data shown in FIG. 10 (a) show a significant induction of Renilla luciferase when cells were exposed to a highly potent genotoxic compound (doxorubicin hydrochloride), less induction of Renilla luciferase when cells were exposed to methylmethane sulfonate, and no induction with phenformin hydrochloride (cytotoxic only) and mannitol (non-toxic). The known genotoxic agents doxorubicin hydrochloride and methylmethane sulfonate have been previously characterized by McCann, J., et al., (1975, PNAS 72, 5135-5139) using the Salmonella/microsome test, as being highly and moderately genotoxic. No Renilla luciferase induction above background was generated when cells were transfected with pGL4.70 MinP [hRLuc] and exposed to the various compounds (see FIG. 10b). These results (10b) acted as an experimental control and highlight the importance and specificity of the p53R2 (RE) pGL4.70 MinP construct for producing results. FIG. 10 (c) shows a significant induction of Renilla luciferase when cells were exposed to a highly potent genotoxic compound (doxorubicin hydrochloride), less induction of Renilla luciferase when cells were exposed to the moderately genotoxic compound, methylmethane sulfonate, and no induction with phenformin hydrochloride (cytotoxic only) and mannitol (non-toxic). No Renilla luciferase induction above background was generated when cells were transfected with pGL4.70 [hRLuc] and exposed to the various compounds (see FIG. 10d). These results (10d) acted as an experimental control and show the importance and specificity of the p53R2 (RE) pGL4.70 construct. The data (FIGS. 10a and 10c) also demonstrate the importance of inclusion of a minimum promoter in the DNA construct (10a) compared with constructs prepared in the absence of the minimum promoter (10c).

The data illustrated in FIG. 10e demonstrate that exposing cells to a known cytotoxic agent (phenformin hydrochloride) results in a reduction in ATP response. There was no reduction in ATP levels with the genotoxic compounds doxorubicin hydrochloride and methylmethane sulfonate indicating that these compounds were not cytotoxic at the concentrations used. These data (10e) are representative of all experiments carried out. Such results are very valuable to researchers who are thus able to predict, using the method described herein, whether an unknown compound is genotoxic, cytotoxic or both genotoxic and cytotoxic.

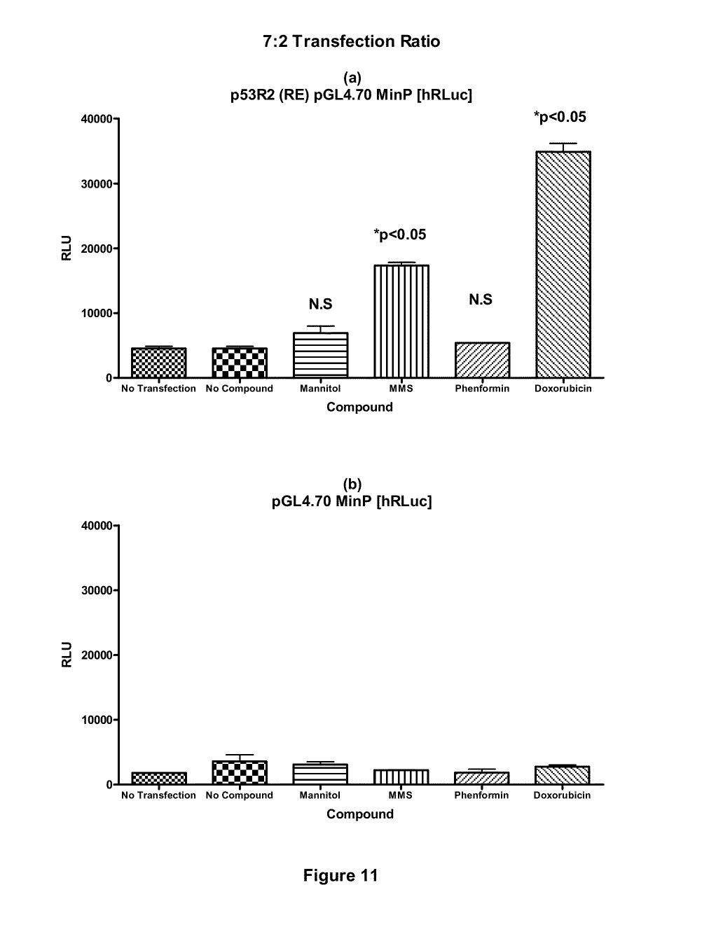

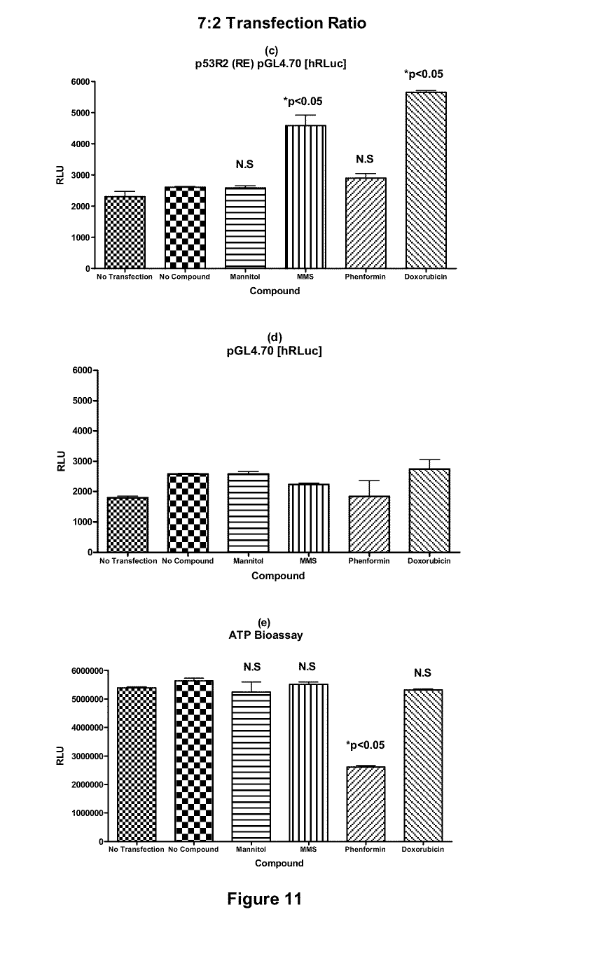

FIG. 11 shows the results of using reduced amounts of transfection reagent (7:2) compared with the data using the higher ratios (8:2) presented in FIG. 10. As can be seen, there is a significantly greater Signal:Background ratio with the higher ratio which increases assay sensitivity.

FIG. 11 depicts results from (a) the p53R2 Renilla Luciferase Reporter Gene Assay. Cells were transfected with 5 μl from a 7:2 transfection complex (7 μl of transfection reagent and 2 μg p53R2 (RE) pGL4.70 MinP [hRLuc]). Transfected HEK 293 cells were treated with known amounts of mannitol (1 mg/ml), phenformin (250 μg/ml), methylmethane sulfonate (50 μg/ml) or doxorubicin hydrochloride (1 μg/ml); (b) control (cells treated as in (a) above, but with pGL4.70 MinP [hRLuc]); (c) p53R2 Renilla Luciferase Reporter Gene Assay. Cells were transfected with 5 μl from a 7:2 transfection complex (7 μl of transfection reagent and 2 μg p53R2 (RE) pGL4.70 [hRLuc]). Transfected HEK 293 cells were treated with known amounts of mannitol, phenformin, methylmethane sulfonate or doxorubicin hydrochloride; (d) control (cells treated as in (c) above, but with pGL4.70 [hRLuc]); (e) ATP Bioluminescent ATP cell viability assay results. The results are shown in RLU and are the mean±1SD (n=3). P<0.05 were significant results as compared with the control (cells only, without treatment with compound) with N.S. indicating no statistically significant difference.

The data in FIG. 11 (a) show a significant induction of Renilla luciferase when cells were exposed to a highly potent genotoxic compound (doxorubicin hydrochloride), less induction of Renilla luciferase when cells were exposed to methylmethane sulfonate, and no induction with phenformin hydrochloride (cytotoxic only) or mannitol (non-toxic). No Renilla luciferase induction above background was generated when cells were transfected with pGL4.70 MinP [hRLuc] and exposed to the various compounds (FIG. 11b). These treatments (e.g. FIG. 11b) acted as an experimental control and highlight the importance and specificity of the p53R2 (RE) pGL4.70 MinP construct.

As can be seen from FIG. 11 (c) there was a significant induction of Renilla luciferase when cells were exposed to a highly potent genotoxic compound (doxorubicin hydrochloride), less induction with methylmethane sulfonate, and no induction with phenformin hydrochloride (cytotoxic only) and mannitol (non-toxic). No Renilla luciferase induction above background was generated when cells were transfected with pGL4.70 [hRLuc] and exposed to the various compounds (FIG. 11d). These treatments acted as an experimental control and show the importance and specificity of the p53R2 (RE) pGL4.70 construct.

The data (FIGS. 11a and 11c) also demonstrate the importance of inclusion of a minimum promoter in the DNA construct (FIG. 11a) compared with constructs prepared in the absence of the minimum promoter (FIG. 11c).

FIG. 11e, which are representative data from all experiments conducted, shows that exposing cells to a known cytotoxic agent (phenformin hydrochloride) results in a reduction in ATP response. There no reduction with the genotoxic compounds doxorubin hydrochloride and methylmethane sulfonate indicating that these latter compounds were not cytotoxic at the concentrations used. Such results are very valuable to researchers who are thus able to predict, using the method described herein, whether an unknown compound is genotoxic, cytotoxic or both genotoxic and cytotoxic.

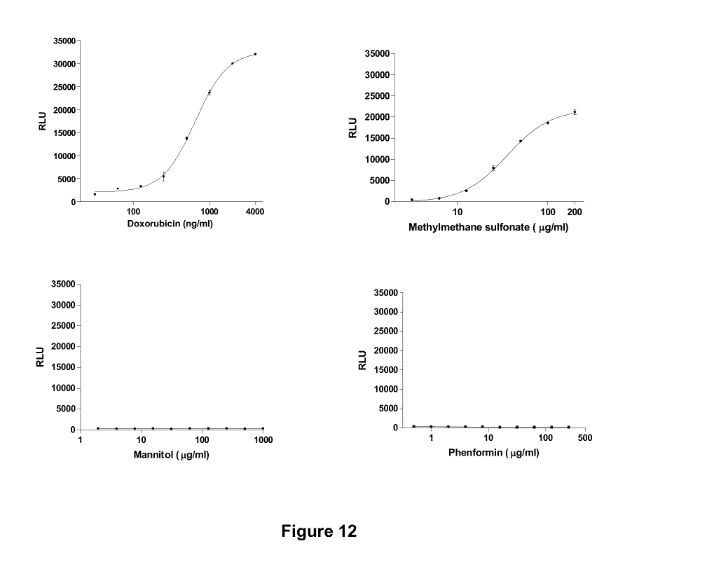

FIG. 12 shows dose-response curves from the p53R2 Renilla Luciferase Reporter Gene Assay. Cells were transfected with 5 μl from an 8:2 transfection complex (8 μl of transfection reagent and 2 μg p53R2 (RE) pGL4.70 MinP [hRLuc]). HEK 293 cells were treated with varying amounts of mannitol, phenformin, methylmethane sulfonate or doxorubicin hydrochloride prior to p53R2 induction. Results are shown in RLU and are shown as a mean±1 SD (n=3). The data presented demonstrate that the assay method described here is capable of detecting varying amounts of genotoxic compounds with diverse potencies.

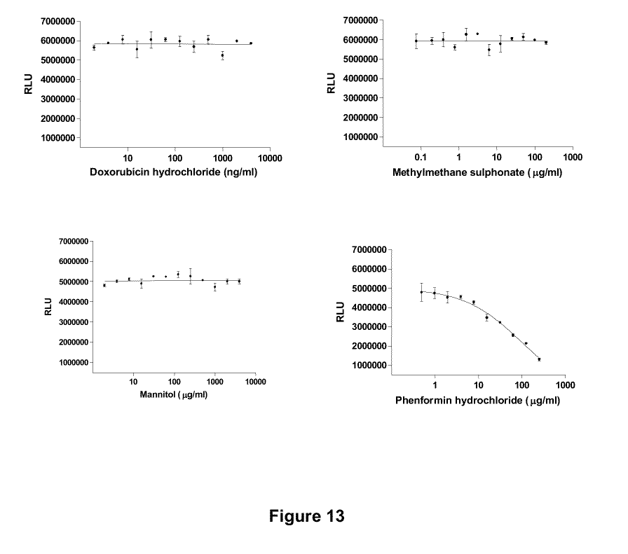

Dose-response curves from the Firefly Luciferase Bioluminescent ATP Cell Viability Assay are presented in FIG. 13. Cells were transfected with 5 μl from an 8:2 transfection complex (8 μl of transfection reagent and 2 μg p53R2 (RE) pGL4.70 MinP [hRLuc]). HEK 293 cells were treated with varying amounts of mannitol, phenformin, methylmethane sulfonate or doxorubicin hydrochloride prior to p53R2 induction followed by ATP measurement. ATP assay results are given in RLU and shown as a mean±1SD (n=3). The data demonstrate that the assay is capable of detecting varying amounts of cytotoxic compounds with diverse potencies.

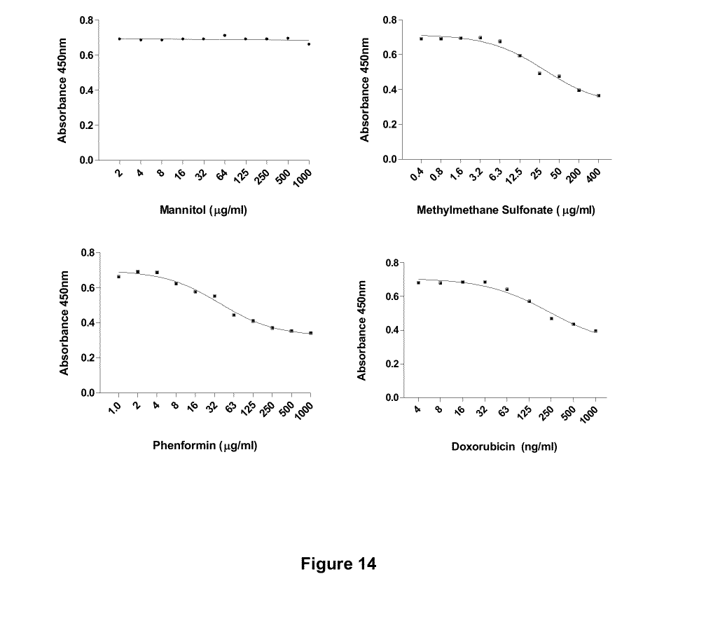

FIG. 14 shows dose-response curves from the Superoxide Dismutase (SOD) (Oxidative Stress) Assay. Cells were transfected with 5 μl from an 8:2 transfection complex (8 μl of transfection reagent and 2 μg p53R2 (RE) pGL4.70 MinP [hRLuc]). HEK 293 cells were treated with varying amounts of mannitol, phenformin, methylmethane sulfonate or doxorubicin hydrochloride prior to p53R2 induction, ATP measurement, (see FIG. 13) followed by superoxide dismutase activity measurement using WST-1 and xanthine oxidase inhibition. Superoxide dismutase assay results are shown in absorbance units at 450 nm. The data demonstrate that the assay is capable of detecting varying amounts of stress-inducing compounds with diverse potencies.

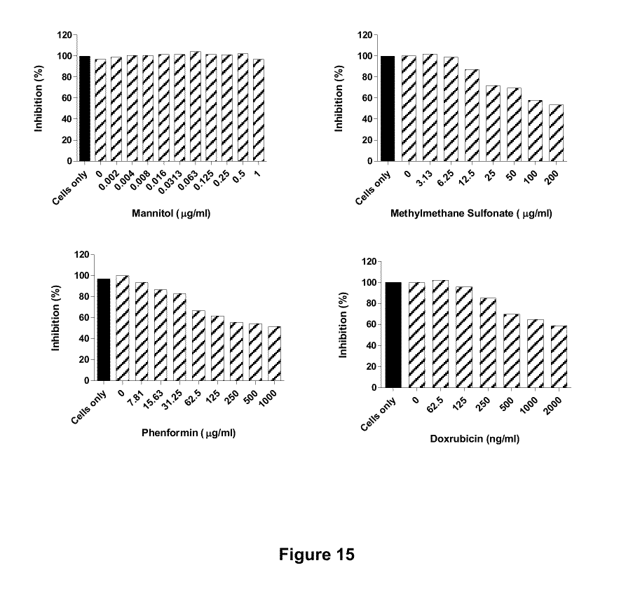

Dose-response curves from the Superoxide Dismutase (SOD) (Oxidative Stress) Assay are also given in FIG. 15. Cells were transfected with 5 μl from an 8:2 transfection complex (8 μl of transfection reagent and 2 μg p53R2 (RE) pGL4.70 MinP [hRLuc]). HEK 293 cells were treated with varying amounts of mannitol, phenformin, methylmethane sulfonate or doxorubicin hydrochloride prior to p53R2 induction (see FIG. 12), ATP measurement, (see FIG. 13) followed by superoxide dismutase activity measurement using WST-1 and xanthine oxidase inhibition. Superoxide dismutase assay results are shown as percentage inhibition using untreated control cells (cells only) as a reference.

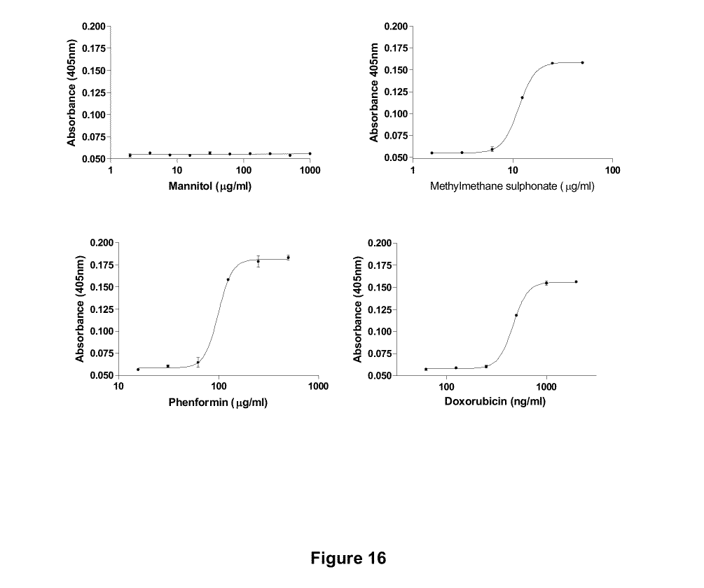

FIG. 16 shows dose-response curves from the Caspase-3 (apoptosis) Assay. Cells were transfected with 5 μl from an 8:2 transfection complex (8 μl of transfection reagent and 2 μg p53R2 (RE) pGL4.70 MinP [hRLuc]). HEK 293 cells were treated with varying amounts of mannitol, phenformin, methylmethane sulfonate or doxorubicin hydrochloride prior to p53R2 induction (see FIG. 12), ATP measurement, (see FIG. 13), superoxide dismutase measurement (see FIG. 15), followed by caspase-3 estimation, using Ac-DEVD-pNA as substrate. Caspase-3 assay results are shown in absorbance units at 405 nm and are reported as a mean±1 SD (n=3). These data demonstrate that the assay is capable of detecting varying amounts of apoptosis-inducing compounds with diverse potencies.

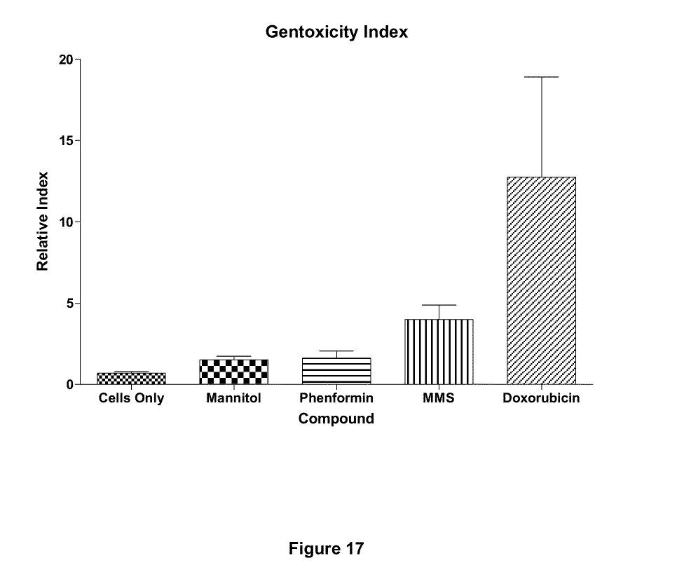

FIG. 17 shows Genotoxicity Index as measured as a function of p53R2 Renilla luciferase activity and ATP measurements. Results are shown as a mean±1 SD (n=3). The genotoxicity index was calculated using 2 μg p53R2 (RE) pGL4.70 MinP [hRLuc] as described above and response measured using Renilla luciferase. ATP was measured using the firefly luciferase assay. Cells were exposed to 1 mg/ml Mannitol, 1 μg/ml doxorubicin, 50 μg/ml methylmethane sulfonate, and 250 μg/ml phenformin. The genotoxicity index was derived by calculation using the following function:—mean genotoxicity response (RLU)÷mean cytotoxicity response (ATP)×10². Results are a relative index of genotoxicity: cytotoxicity.

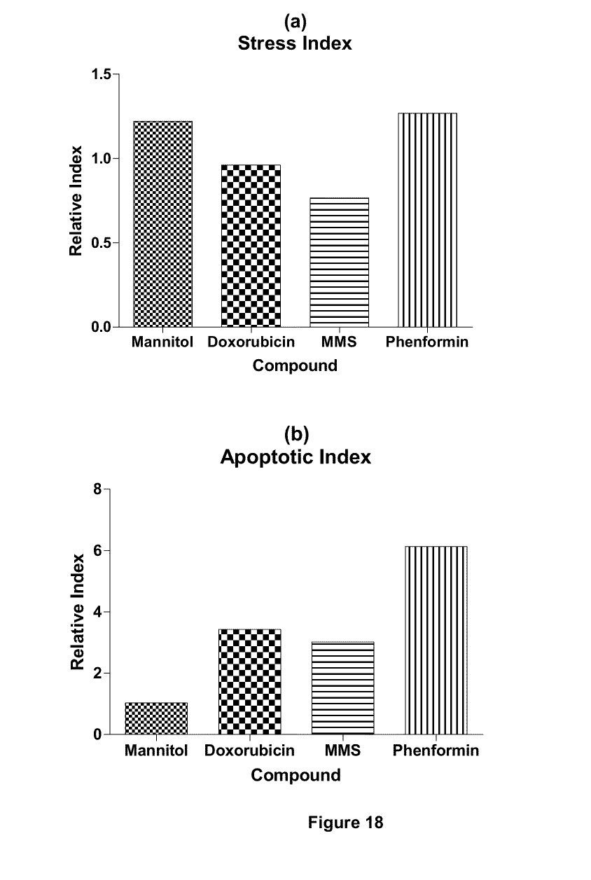

FIG. 18 shows (a) Cellular Stress Index as measured as a function of superoxide dismutase activity and ATP measurement, and, (b) Apoptotic Index as measured as a function of caspase-3 activity and ATP measurement. The cellular stress index (a) was calculated the Superoxide Dismutase Assay as described above and response measured at 450 nm. ATP was measured using the firefly luciferase assay. Cells were exposed to 1 mg/ml Mannitol, 1 μg/ml doxorubicin, 50 μg/ml methylmethane sulfonate, and, 250 μg/ml phenformin. The cellular stress index was derived by calculation using the following function:—mean superoxide response (at 450 nm)÷mean cytotoxicity response (ATP)×10⁷. Results are a relative index of cellular stress: cytotoxicity.

The apoptotic index (b) was calculated the Caspase-3 Assay as described in the text, and, response measured at 405 nm. ATP was measured using the firefly luciferase assay. Cells were exposed to 1 mg/ml Mannitol, 1 μg/ml doxorubicin, 50 μg/ml methylmethane sulfonate, and, 250 μg/ml phenformin. The apoptotic index was derived by calculation using the following function:—mean apoptotic response (at 405 nm)÷mean cytotoxicity response (ATP)×10⁸. Results are a relative index of apoptosis: cytotoxicity.

TABLE 1
Genotoxicity Index as measured as a function of p53R2 Renilla
luciferase activity and ATP measurements. The Genotoxicity
index was derived by calculation using the following function:
mean genotoxicity response (Renilla RLU) ÷ mean cytotoxicity
response (ATP) × 10². Results are a relative index of
genotoxicity: cytotoxicity.

| | Mean Genotoxic | Standard |
|---|---|---|
| Compound | Index | Deviation |
| Cells only control | 0.70 | 0.14 |
| Mannitol | 1.52 | 0.31 |
| Doxorubicin | 12.74 | 4.73 |
| Methylmethane sulfonate | 4.01 | 1.23 |
| Phenformin | 1.63 | 0.62 |

The known genotoxic agents, doxorubicin and methylmethane sulfonate, were characterized by McCann, J., et al., (1975, PNAS 72, 5135-5139) using the Salmonella/microsome test as being highly and moderately genotoxic. This characterization was confirmed as both chemicals exhibit high Genotoxic Index values. Of the chemicals used in this study the highly genotoxic doxorubicin exhibited the highest Genotoxic Index (12.74). The cytotoxic compound phenformin exhibited a low genotoxic response (see FIGS. 10 & 11) and a low Genotoxic Index value confirming that it is not genotoxic. The low phenformin Genotoxic Index value was derived from the low genotoxic and low ATP response of the cells (as described in FIGS. 10 and 11). In the Cell-Titer Glo Luciferase ATP Quantification assay system, low ATP levels are indicative of cellular death. The Genotoxic Index value exhibited by phenformin was comparable to that exhibited by mannitol (which is known to be non-genotoxic and non-cytotoxic). A simple comparison between the ATP values differentiates between the cytotoxic phenformin and mannitol (see FIGS. 10 & 11).

Therefore these data indicate that for the compounds studied the Genotoxic Index is able to categorize clearly between genotoxic and cytotoxic compounds e.g. doxorubicin and phenformin. In combination with absolute ATP levels, the Genotoxic Index differentiates between control (mannitol) and cytotoxic (phenformin) compounds

TABLE 2
Cellular stress Index as measured as a function of superoxide
dismutase activity and ATP measurement. The cellular stress
index was derived by calculation using the following function:
mean superoxide dismutase response ÷ mean cytotoxicity
response (ATP) × 10⁷. Results are a relative index of
cellular stress: cytotoxicity.

| | Mean Stress | Standard |
|---|---|---|
| Compound | Index | Deviation |
| Mannitol | 1.22 | 0.13 |
| Doxorubicin | 0.96 | 0.02 |
| Methylmethane | 0.77 | 0.03 |
| sulfonate | | |
| Phenformin | 1.27 | 0.06 |

The genotoxic compounds doxorubicin and methylmethane sulfonate exhibit the lowest Cellular Stress Index values compared to the control compound mannitol. These values are derived from cells that exhibit comparable ATP levels (see FIGS. 10 and 11). Therefore the Cellular Stress Index indicates that although not cytotoxic doxorubicin and methylmethane sulfonate may actually cause a degree of cellular stress compared to the non-cytotoxic mannitol.

The Cellular Stress Index exhibited by cells exposed to phenformin is comparable to that exhibited by cells exposed to mannitol. Inspection of the ATP levels derived from these cells indicates clearly that those exposed to phenformin are undergoing cell death and therefore the comparable Cellular Stress Index values can be differentiated by referral to ATP levels.

TABLE 3
Apoptotic Index as measured as a function of caspase-3 activity and
ATP measurement. The apoptotic index was derived by calculation
using the following function: mean apoptotic response (as determined
by the Caspase-3 assay) ÷ mean cytotoxicity response (ATP) × 10⁸.
Results are a relative index of apoptosis: cytotoxicity.

| | Mean Apoptotic | Standard |
|---|---|---|
| Compound | Index | Deviation |
| Mannitol | 1.04 | 0.17 |
| Doxorubicin | 3.43 | 0.15 |
| Methylmethane | 3.02 | 0.71 |
| sulfonate | | |
| Phenformin | 6.13 | 0.08 |

The cytotoxic compound phenformin exhibits the highest apoptotic index indicating that a number of cells when exposed to phenformin are probably entering apoptosis. The high Apoptotic Index is in the presence of a low ATP value which is a reflection to cellular death (see FIGS. 10 and 11). The Apoptotic Index value for the genotoxic compounds doxorubicin and methylmethane sulfonate are lower than that exhibited by phenformin but are higher than that exhibited by mannitol.

Doxorubicin and methylmethane sulfonate are not cytotoxic (as based upon ATP levels, see FIGS. 10 and 11). A measurement based solely upon apoptosis values (as described in FIG. 16) indicates that doxorubicin, methylmethane sulfonate and phenformin all exhibit comparable caspase 3 activities. These data could be interpreted that all three compounds are cytotoxic. However by incorporating ATP levels as a measured of cell viability the Apoptotic Index is able to differentiate between the cytotoxic/apoptotic activity of phenformin and the non-cytotoxic activities of doxorubicin and methylmethane sulfonate.

The doxorubicin and methylmethane sulfonate Apoptotic Indices are elevated compared to that exhibited by cells exposed to mannitol. which are the lowest generated in the study. This may indicate that a percentage of cells exposed to these genotoxic chemicals are undergoing apoptosis.

Whilst the present invention has been described in connection with various embodiments, those skilled in the art will be aware that many different embodiments and variations are possible. All such variations and embodiments are intended to fall within the scope of the present invention as defined by the appended claims.

1. A multiplex method for determining the toxicity of an agent on a population of eukaryotic cells, wherein said population of cells optionally comprises a nucleic acid construct comprising a DNA damage induced response element operably linked to a sequence encoding a reporter gene,
said method comprising
i) contacting a first population of eukaryotic cells in a test vessel with an agent to produce a test sample and contacting a second population of said cells in a control vessel with a control treatment to produce a control sample;
ii) lysing the cells in both the test sample and the control sample
iii) quantifying the levels of ATP present in both the test sample and the control sample, wherein a difference in said levels of ATP between the samples is indicative of the cytotoxicity of the agent; and
iv) measuring the activity of an endogenous enzyme or reporter protein in both the test sample and the control sample, wherein a difference in the activities between the samples is indicative of the oxidative stress, apoptotic activity or genotoxicity induced by the agent;
wherein steps i) to iv) are carried out on the same test sample in the same test vessel and on the same control sample in the same control vessel.

2. The method of claim 1, wherein a luciferase enzyme is added to quantify the levels of ATP in step iii).

3. The method of claim 1, wherein the activity of an oxidative enzyme is measured in step iv) as indicative of the oxidative stress induced by the agent.

4. (canceled)

5. The method of claim 1, wherein the activity of a proteolytic enzyme is measured in step iv) as indicative of the apoptotic activity of the agent.

6-7. (canceled)

8. The method of claim 1, wherein the test vessel and the control vessel are wells in a microwell plate.

9. The method of claim 1, wherein the population of cells comprises a nucleic acid construct comprising a DNA damage induced response element operably linked to a sequence encoding a reporter gene, the method comprising the step of measuring the activity of the reporter gene protein product in both the test sample and the control sample, wherein a difference in said activity between the samples is indicative of the genotoxicity of the agent.

10. The method of claim 9, additionally comprising the step of adding a substrate of the reporter gene protein product to both said test sample and said control sample under conditions to permit expression of the reporter gene prior to measuring said activity in both the test sample and the control sample.

11. (canceled)

12. The method of claim 9, wherein said DNA damage induced response element is located within a group of promoters selected from the group consisting of p53R2, GADD45α, mrt-2, hus-1, rad-5, cep-1, egl-1, ape-1, abl-1, brc-1, brd-1, pme-5, kin-2-, hpr-9, hpr-17, chk-1 and chk-2.

13. The method of claim 12, wherein the DNA damage induced response element is located within the p53R2 promoter.

14. The method of claim 9, wherein said nucleic acid construct additionally comprises a promoter sequence.

15. The method of claim 14, wherein said promoter sequence is selected from the group of promoters consisting of p53R2, GADD45α, mrt-2, hus-1, rad-5, cep-1, egl-1, ape-1, abl-1, brc-1, brd-1, pme-5, kin-2-, hpr-9, hpr-17, chk-1 and chk-2

16. The method of claim 14, wherein the promoter is the p53R2 promoter.

17. The method of claim 9, wherein the reporter gene is selected from the group consisting of a fluorescent protein, a luciferase, a β-lactamase, a dihydrofolate reductase, a β-glucuronidase, a β-galactosidase, a chloramphenicol acetyltransferase, a ubiquitinase, an alkaline phosphatase, a tryptophan synthase reporter gene and a nitro reductase reporter gene.

18-19. (canceled)

20. The method of claim 19, wherein the DNA damage induced response element is derived from the p53R2 promoter, the reporter gene is a Renilla luciferase, and a firefly luciferase enzyme is added to quantify the levels of ATP in step iii).

21-24. (canceled)

25. The method of claim 1, wherein the agent is an organic or inorganic compound.

26. The method of claim 1, further comprising correlating the cytotoxicity of the agent with any or all of the genotoxicity, oxidative stress or apoptotic inducing activities of the agent.

27. (canceled)

28. A kit comprising a population of cells and instructions for carrying out the method of claim 1.

29. A kit comprising a population of cells comprising a nucleic acid construct comprising a DNA damage induced response element operably linked to a sequence encoding a reporter gene and instructions for carrying out the method of claim 1.

30. A kit comprising a vector comprising a nucleic acid construct comprising a DNA damage induced response element operably linked to a sequence encoding a reporter gene and instructions for carrying out the method of claim 1.

31. (canceled)
